(19) United States
(12) Patent Application Publication
(10) Pub. No.: US 2004/0042974 A1
(43) Pub. Date: Mar. 4, 2004

(54) Two-compartment aerosol device comprising a hair-styling aqueous composition and hair-styling method

(75) Inventors: Christine Dupuis, Paris (FR); Isabelle Rollat, Paris (FR); Katarina Benabdillah, Clichy (FR); Henri Samain, Bievres (FR); Jonathan Gawtrey, Boulogne (FR)

Correspondence Address: STEPTOE & JOHNSON LLP, 1330 Connecticut Avenue, NW, Washington, DC, 20036-1795, US

(73) Assignee: L'OREAL, Paris (FR)

(21) Appl. No.: 10/448,421

(22) Filed: May 30, 2003

Related U.S. Application Data
(60) Provisional application No. 60/385,589, filed on Jun. 5, 2002.

(30) Foreign Application Priority Data
May 31, 2002 (FR) 0206735

(51) Int. Cl. A61K 7/00; B65D 83/06

(52) U.S. Cl. 424/47; 222/394

(57) ABSTRACT

The present invention relates to a two-compartment aerosol device comprising, in a first compartment, a hair-styling aqueous composition, and, in a second compartment, a compressed gas chosen from air, nitrogen, carbon dioxide and mixtures thereof. The said hair-styling composition comprises less than 5% by weight, relative to the total weight of the said composition, of C₁-C₄ monohydric alcohols, and at least one water-soluble or water-dispersible fixing polymer, the said water-soluble or water-dispersible fixing polymer being different from a vinylpyrrolidone homopolymer.

This device preferably constitutes a hair lacquer.

The invention also relates to a hair-styling method using the device according to the invention.

The present invention relates to a two-compartment aerosol device comprising a hair-styling aqueous composition and a compressed gas, and to a hair-styling method.

Hair-styling compositions, such as lacquers and sprays, packaged in aerosol spray form are generally composed of a liquid phase comprising, in a cosmetically acceptable alcoholic or aqueous-alcoholic medium, at least one film-forming polymer, and of a propellant which is a liquefied gas under reduced pressure or dissolved in the liquid phase.

In the field of hair products, it is sought to manufacture aerosol lacquers containing no volatile organic compound such as ethanol or dimethyl ether, this being essentially for ecological reasons, while preserving good properties of shaping and retaining the hairstyle.

The document U.S. Pat. No. 5,626,840 describes hair fixing compositions which may be in aerosol form. They comprise a polyurethane in an aqueous or aqueous-alcoholic medium, an inorganic or organic base, and a solvent which may be water or a mixture of water and a polar organic solvent.

This document describes a marked decrease in volatile organic compounds in hair-styling compositions.

The applicant has surprisingly discovered that the use of a two-compartment device, comprising, in a first compartment, a hair-styling aqueous composition and, in a second compartment, a compressed gas made it possible to obtain good spraying conditions and a lacquer having shaping and retention properties which are entirely equivalent to those of state of the art lacquers and additionally not containing any volatile organic compound.

The said properties are obtained in particular when the hair-styling composition comprises at least one water-soluble or water-dispersible fixing polymer which is different from vinylpyrrolidone homopolymers.

The subject of the present invention is therefore a two-compartment aerosol device comprising, in a first compartment, a hair-styling aqueous composition as described below, and, in a second compartment, a compressed gas as described below.

Another subject of the present invention consists in a hair-styling method using the device of the invention.

Other subjects, characteristics, features and advantages of the invention will emerge even more clearly on reading the description and the various examples which follow.

The two-compartment aerosol device of the invention comprises

a) in a first compartment, a hair-styling aqueous composition comprising less than 5% by weight, relative to the total weight of the said composition, of C₁-C₄ monohydric alcohols, and at least one water-soluble or water-dispersible fixing polymer, and

b) in a second compartment, a compressed gas chosen from air, nitrogen, carbon dioxide and mixtures thereof, air being particularly preferred,

with the proviso that the water-soluble or water-dispersible fixing polymer is not a vinylpyrrolidone homopolymer.

The said compressed gas is preferably used under a pressure of between 1 and 12 bar, better still between 9 and 11 bar.

The said hair-styling composition preferably comprises less than 2% by weight, and still more preferably 0% by weight of C₁-C₄ monohydric alcohols.

The expression water-soluble polymer is understood to mean, for the purposes of the present invention, polymers which, when introduced into water at 25° C., at a concentration by weight equal to 0.1%, as they are or in neutralized form, make it possible to obtain a macroscopically homogeneous and transparent solution or suspension, that is to say having a light transmittance value, at a wavelength equal to 500 nm, through a sample 1 cm thick, of at least 70%, preferably of at least 80%.

The expression water-dispersible polymer is understood to mean, for the purposes of the present invention, polymers which, when introduced into water at 25° C., at a concentration by weight equal to 0.1%, give a dispersion of insoluble particles. It is possible to use latexes or pseudolatexes in particular.

The use of these polymers in the device of the invention also makes it possible to avoid a phenomenon of blocking of the spray nozzles by the composition.

The fixing polymers suitable in the invention are chosen in particular from cationic, anionic, amphoteric and nonionic polymers, and mixtures thereof.

The cationic fixing polymers which can be used according to the present invention are preferably chosen from polymers containing primary, secondary, tertiary and/or quaternary amine groups which form part of the polymer chain or which are directly attached thereto, and having a number-average molecular weight of between 500 and about 5,000,000, and preferably between 1,000 and 3,000,000.

Among these polymers, there may be mentioned more particularly the following cationic polymers:

(1) acrylic or methacrylic ester or amide homopolymers or copolymers with amino functional groups and comprising at least one of the units of the following formulae:

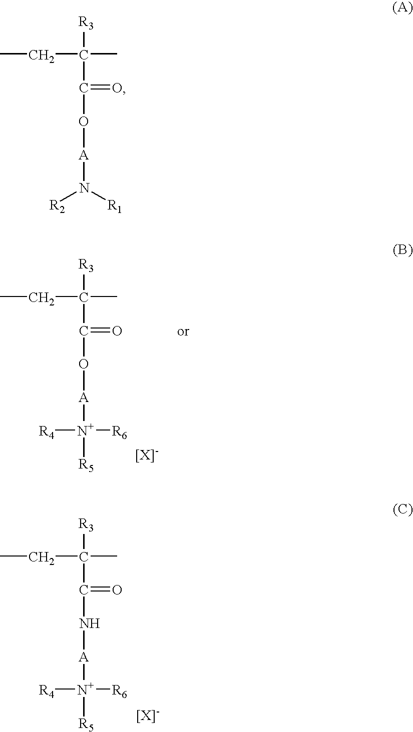

in which:

R₁ and R₂, which are identical or different, each represent a hydrogen atom or an alkyl group having from 1 to 6 carbon atoms;

R₃ denotes a hydrogen atom or a CH₃ group;

A is a linear or branched alkyl group of 1 to 6 carbon atoms or a hydroxyalkyl group of 1 to 4 carbon atoms;

R₄, R₅, R₆, which are identical or different, represent an alkyl group having from 1 to 18 carbon atoms or a benzyl group;

X denotes a methosulphate anion or a halide such as chloride or bromide.

The copolymers of the family (1) contain, in addition, one or more units derived from comonomers which may be chosen from the family of acrylamides, methacrylamides, diacetone acrylamides, acrylamides and methacrylamides substituted on the nitrogen with lower (C₁-C₄)alkyl groups, groups derived from acrylic or methacrylic acids or esters thereof, vinyllactams such as vinylpyrrolidone or vinylcaprolactam, vinyl esters.

Thus, among these copolymers of the family (1), there may be mentioned:

the copolymers of acrylamide and dimethylamino-ethyl methacrylate quaternized with dimethyl sulphate or with a dimethyl halide such as that sold under the name HERCOFLOC® by the company HERCULES,

the copolymers of acrylamide and methacryloyloxy-ethyltrimethylammonium chloride described, for example, in Patent Application EP-A-080976 and sold under the name BINA QUAT P 100 by the company CIBA GEIGY,

the copolymers of acrylamide and methacryloyloxy-ethyltrimethylammonium methosulphate such as those sold under the name RETEN by the company HERCULES,

the vinylpyrrolidone/dialkylaminoalkyl acrylate or methacrylate copolymers, quaternized or otherwise, such as the products sold under the name “GAFQUAT®” by the company ISP such as for example “GAFQUAT® 734” or “GAFQUAT® 755” or alternatively the products called “COPOLYMER® 845, 958 and 937”. These polymers are described in detail in French Patents 2,077,143 and 2,393,573,

the dimethylaminoethyl methacrylate/vinylcaprolactam/vinyl pyrrolidone terpolymers such as the product sold under the name GAFFIX® VC 713 by the company ISP,

and the quaternized vinylpyrrolidone/dimethyl-aminopropyl methacrylamide copolymers such as the product sold under the name “GAFQUAT® HS 100” by the company ISP;

(2) cationic polysaccharides, preferably with quaternary ammonium, such as those described in U.S. Pat. Nos. 3,589,578 and 4,031,307 such as guar gums containing cationic trialkylammonium groups. Such products are marketed in particular under the trade names JAGUAR C13 S, JAGUAR C 15, JAGUAR C 17 by the company MEYHALL;

(3) quaternary copolymers of vinylpyrrolidone and vinylimidazole;

(4) chitosans or their salts; the salts which can be used are in particular chitosan acetate, lactate, glutamate, gluconate or pyrrolidonecarboxylate.

Among these compounds, there may be mentioned chitosan having a deacetylation value of 90.5% by weight, sold under the name KYTAN BRUT STANDARD by the company ABER TECHNOLOGIES, chitosan pyrrolidonecarboxylate marketed under the name KYTAMER® PC by the company AMERCHOL.

(5) cationic cellulose derivatives such as cellulose copolymers or cellulose derivatives copolymers grafted with a quaternary ammonium-containing water-soluble monomer, and described especially in U.S. Pat. No. 4,131,576, such as hydroxyalkyl celluloses like hydroxymethyl, hydroxyethyl or hydroxypropyl celluloses grafted especially with a methacryloyloxyethyltrimethylammonium, methacrylamidopropyltrimethylammonium or dimethyldiallylammonium salt.

The commercialized products corresponding to this definition are more particularly the products sold under the name “Celquat L 200” and “Celquat H 100” by the company National Starch.

The anionic fixing polymers generally used are polymers containing groups derived from carboxylic, sulphonic or phosphoric acid and have a number-average molecular weight of between about 500 and 5,000,000.

The carboxylic groups are provided by unsaturated mono- or dicarboxylic acid monomers such as those corresponding to the formula:

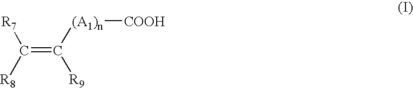

in which n is an integer from 0 to 10, A₁ denotes a methylene group, optionally linked to the carbon atom of the unsaturated group or to the neighbouring methylene group, when n is greater than 1, through a heteroatom such as oxygen or sulphur, R₇ denotes a hydrogen atom, or a phenyl or benzyl group, R₈ denotes a hydrogen atom, or a carboxyl or lower alkyl group, R₉ denotes a hydrogen atom or a lower alkyl group, a group —CH₂—COOH, or a phenyl or benzyl group.

In the above-mentioned formula, a lower alkyl group denotes preferably a group having 1 to 4 carbon atoms, and in particular methyl and ethyl groups.

The anionic fixing polymers with carboxylic groups preferred according to the invention are:

A) the homo- or copolymers of acrylic or methacrylic acid or their salts and in particular the products sold under the names VERSICOL® E or K by the company ALLIED COLLOID and ULTRAHOLD® by the company BASF, the copolymers of acrylic acid and of acrylamide sold in the form of their sodium salts under the names RETEN 421, 423 or 425 by the company HERCULES, the sodium salts of the polyhydroxycarboxylic acids;

B) the copolymers of acrylic or methacrylic acids with a monoethylene monomer such as ethylene, styrene, vinyl esters, acrylic or methacrylic acid esters, optionally grafted onto a polyalkylene glycol such as polyethylene glycol and optionally crosslinked. Such polymers are described in particular in French Patent 1,222,944 and German Application 2,330,956, copolymers of this type containing in their chain an acrylamide unit optionally N-alkylated and/or hydroxyalkylated as described especially in Luxembourg Patent applications 75370 and 75371 or offered under the name QUADRAMER by the company AMERICAN CYANAMID. There may also be mentioned the copolymers of acrylic acid and C₁-C₄ alkyl methacrylate and the terpolymers of vinylpyrrolidone, acrylic acid and C₁-C₂₀ alkyl, for example lauryl, methacrylate such as that sold by the company ISP under the name ACRYLIDONE® LM and the methacrylic acid/ethyl acrylate/tert-butyl acrylate terpolymers such as the product sold under the name LUVIMER® 100 P by the company BASF.

There may also be mentioned the methacrylic acid/acrylic acid/ethyl acrylate/methyl methacrylate copolymers in aqueous dispersion, marketed under the name AMERHOLD® DR 25 by the company AMERCHOL.

C) the copolymers of crotonic acid such as those containing in their chain vinyl propionate or acetate units and optionally other monomers such as methallyl or allyl esters, vinyl ether or vinyl ester of a linear or branched saturated carboxylic acid with a long hydrocarbon chain such as those containing at least 5 carbon atoms, it being possible for these polymers to be optionally grafted or crosslinked or alternatively another vinyl, allyl or methallyl ester monomer of an α- or β-cyclic carboxylic acid. Such polymers are described, inter alia, in French Patents 1,222,944; 1,580,545; 2,265,782; 2,265,781; 1,564,110 and 2,439,798. Commercial products entering into this class are the resins 28-29-30, 26-13-14 and 28-13-10 sold by the company NATIONAL STARCH.

D) the copolymers derived from C₄-C₈ monounsaturated carboxylic acids or anhydrides chosen from:

the copolymers comprising (i) one or more itaconic, fumaric or maleic acids or anhydrides and (ii) at least one monomer chosen from vinyl esters, vinyl ethers, vinyl halides, phenylvinyl derivatives, acrylic acid and its esters, the anhydride functions of these copolymers being optionally monoesterified or monoamidated. Such polymers are described in particular in patents U.S. Pat. Nos. 2,047,398; 2,723,248; 2,102,113, Patent GB 839,805. The commercialized products are especially those sold under the names GANTREZ® AN or ES by the company ISP.

the copolymers comprising (i) one or more maleic, citraconic or itaconic anhydride units and (ii) one or more monomers chosen from allyl or methallyl esters optionally containing one or more acrylamide, methacrylamide, α-olefin, acrylic or methacrylic ester, acrylic or methacrylic acid or vinylpyrrolidone groups in their chain, the anhydride functions of these copolymers being optionally monoesterified or monoamidated.

These polymers are, for example, described in French Patents 2,350,384 and 2,357,241 by the applicant.

E) The polyacrylamides containing carboxylate groups.

The homopolymers and copolymers comprising sulphonic groups are polymers containing vinylsulphonic, styrenesulphonic, naphthalenesulphonic or acrylamidoalkylsulphonic units.

These polymers may be especially chosen from:

the salts of polyvinylsulphonic acid having a molecular weight of between about 1,000 and 100,000 as well as the copolymers with an unsaturated comonomer such as acrylic or methacrylic acids and their esters as well as acrylamide or its derivatives, vinyl ethers and vinylpyrrolidone;

the salts of polystyrenesulphonic acid, such as the sodium salts sold for example under the names Flexan® 500 and Flexan® 130 by National Starch. These compounds are described in Patent FR 2,198,719.

the salts of polyacrylamidesulphonic acids, such as those mentioned in patent U.S. Pat. No. 4,128,631 and more particularly polyacrylamidoethylpropanesulphonic acid sold under the name COSMEDIA POLYMER HSP 1180 by Henkel.

According to the invention, the anionic fixing polymers are preferably chosen from the acrylic acid copolymers such as the acrylic acid/ethyl acrylate/N-tert-butylacrylamide terpolymers sold under the name ULTRAHOLD® STRONG by the company BASF, the copolymers derived from crotonic acid such as the vinyl acetate/vinyl tert-butylbenzoate/crotonic acid terpolymers and the crotonic acid/vinyl acetate/vinyl neododecanoate terpolymers sold under the name Resine 28-29-30 by the company NATIONAL STARCH, the polymers derived from itaconic, fumaric and maleic acids or anhydrides with vinyl esters, vinyl ethers, vinyl halides, phenylvinyl derivatives, acrylic acid and its esters such as the monoesterified maleic anhydride/methylvinyl ether copolymers sold under the name GANTREZ® by the company ISP, the copolymers of methacrylic acid and of methyl methacrylate sold under the name EUDRAGIT® L by the company ROHM PHARMA, the copolymers of methacrylic acid and of ethyl acrylate sold under the name LUVIMER® MAEX or MAE by the company BASF and the vinyl acetate/crotonic acid copolymer sold under the name LUVISET CA 66 by the company BASF and the vinyl acetate/crotonic acid copolymer grafted with polyethylene glycol sold under the name ARISTOFLEX® A by the company BASF.

Among the above-cited anionic fixing polymers, it is more particularly preferable to use in the context of the present invention the monoesterified maleic anhydride/methylvinyl ether copolymers sold under the name GANTREZ® ES 425 by the company ISP, the terpolymers acrylic acid/ethyl acrylate/N-tert-butylacrylamide sold under the name ULTRAHOLD® STRONG by the company BASF, the copolymers of methacrylic acid and of methyl methacrylate sold under the name EUDRAGIT® L by the company ROHM PHARMA, the vinyl acetate/vinyl tert-butylbenzoate/crotonic acid terpolymers and the crotonic acid/vinyl acetate/vinyl neododecanoate terpolymers sold under the name Resine 28-29-30 by the company NATIONAL STARCH, the copolymers of methacrylic acid and ethyl acrylate sold under the name LUVIMER® MAEX or MAE by the company BASF, the vinylpyrrolidone/acrylic acid/lauryl methacrylate terpolymers sold under the name ACRYLIDONE® LM by the company ISP.

The fixing amphoteric polymers which can be used in accordance with the invention may be chosen from the polymers containing B and C units statistically distributed in the polymer chain where B denotes a unit which is derived from a monomer containing at least one basic nitrogen atom and C denotes a unit which is derived from an acidic monomer containing one or more carboxylic or sulphonic groups or alternatively B and C may denote groups which are derived from carboxybetaines or sulphobetaines zwitterionic monomers;

B and C may also denote a cationic polymer chain containing primary, secondary, tertiary or quaternary amine groups, in which at least one of the amine groups carries a carboxylic or sulphonic group linked via a hydrocarbon group or alternatively B and C form part of a chain of a polymer with an α,β-dicarboxylic ethylene unit in which one of the carboxylic groups has been caused to react with a polyamine containing one or more primary or secondary amine groups.

The amphoteric fixing polymers corresponding to the above-given definition, which are more particularly preferred, are chosen from the following polymers:

(1) The copolymers with acidic vinyl units and with basic vinyl units such as those resulting from the copolymerization of a monomer derived from a vinyl compound carrying a carboxylic group, such as more particularly acrylic acid, methacrylic acid, maleic acid, alpha-chloroacrylic acid, and of a basic monomer derived from a substituted vinyl compound containing at least one basic atom, such as more particularly dialkylaminoalkyl methacrylate and acrylate, dialkylaminoalkylmethacrylamide and acrylamide. Such compounds are described in U.S. Pat. No. 3,836,537.

(2) The polymers containing units which are derived from:

a) at least one monomer chosen from acrylamides or methacrylamides substituted on the nitrogen atom by an alkyl group,

b) at least one acidic comonomer containing one or more reactive carboxylic groups, and

c) at least one basic comonomer such as acrylic and methacrylic acid esters with primary, secondary, tertiary and quaternary amine substituents and the product of quaternization of dimethylaminoethyl methacrylate with dimethyl or diethyl sulphate.

The most particularly preferred N-substituted acrylamides or methacrylamides according to the invention are compounds whose alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide as well as the corresponding methacrylamides.

The acidic comonomers are more particularly chosen from acrylic, methacrylic, crotonic, itaconic, maleic and fumaric acids as well as the C₁-C₄ alkyl monoesters of maleic or fumaric anhydrides or acids.

The preferred basic comonomers are aminoethyl, butylaminoethyl, N,N′-dimethylaminoethyl, N-tert-butylaminoethyl methacrylates.

Particularly used are the copolymers whose CTFA name (4th ed. 1991) is Octylacrylamide/acrylates/butylaminoethyl methacrylate copolymer such as the products sold under the name AMPHOMER® or LOVOCRYL® 47 by the company NATIONAL STARCH.

(3) The partially or completely acylated and crosslinked polyaminoamides derived from polyaminoamides of general formula:

[CO—R₁₀CO-Z] (I)

in which R₁₀ represents a divalent radical derived from a saturated dicarboxylic acid, a mono- or dicarboxylic aliphatic acid with ethylenic double bond, an ester of a lower alkanol having 1 to 6 carbon atoms and of these acids, or a radical which is derived from the addition of any one of the said acids with a bis-primary or bis-secondary amine, and Z denotes a radical of a bis-primary, mono- or bis-secondary polyalkylene-polyamine and preferably represents:

a) in the proportions of 60 to 100 mol %, the radical

—NH[(CH₂)ₓ—NH]ₚ (IV)

where x=2 and p=2 or 3, or alternatively x=3 and p=2

this radical being derived from the diethylenetriamine, triethylenetetraamine or dipropylenetriamine;

b) in the proportions of 0 to 40 mol %, the above radical (IV) in which x=2 and p=1 and which is derived from ethylenediamine, or the radical which is derived from piperazine:

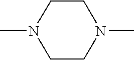

c) in the proportions of 0 to 20 mol %, the radical —NH—(CH₂)₆—NH— which is derived from hexamethylenediamine,

these polyaminoamides being crosslinked by adding a bifunctional crosslinking agent chosen from the epihalohydrins, diepoxides, dianhydrides, bis-unsaturated derivatives, by means of 0.025 to 0.35 mol of crosslinking agent per amine group of the polyaminoamide and acylated by the action of acrylic acid, chloroacetic acid or of an alkanesultone or of their salts.

The saturated carboxylic acids are preferably chosen from the acids having 6 to 10 carbon atoms such as adipic, 2,2,4-trimethyladipic and 2,4,4-trimethyladipic acids, terephthalic acid, the acids with ethylene double bond such as for example acrylic, methacrylic and itaconic acids.

The alkanesultones used in the acylation are preferably propane or butanesultone, the salts of the acylating agents are preferably the sodium or potassium salts.

4) The polymers containing zwitterionic units of formula:

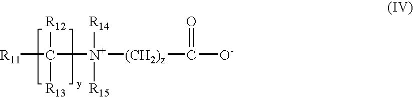

in which R₁₁ denotes a polymerizable unsaturated group such as an acrylate, methacrylate, acrylamide or methacrylamide group, y and z represent an integer from 1 to 3, R₁₂ and R₁₃ represent a hydrogen atom, methyl, ethyl or propyl, R₁₄ and R₁₅ represent a hyrogen atom or an alkyl radical such that the sum of the carbon atoms in R₁₄ and R₁₅ does not exceed 10.

The polymers comprising such units may also comprise units derived from non-zwitterionic monomers such as dimethyl- or diethylaminoethyl acrylate or methacrylate or alkyl acrylates or methacrylates, acrylamides or methacrylamides or vinyl acetate.

By way of example, there may be mentioned the methyl methacrylate/methyl dimethylcarboxymethylammonioethyl methacrylate copolymer such as the product sold under the name DIAFORMER Z301 by the company SANDOZ.

5) The polymers derived from chitosan containing monomeric units corresponding to the following formulae:

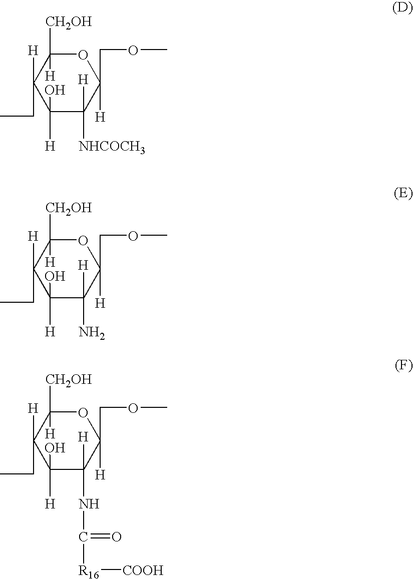

the D unit being present in proportions of between 0 and 30%, the E unit in proportions of between 5 and 50% and the F unit in proportions of between 30 and 90%, it being understood that in this F unit, R₁₆ represents a radical of formula:

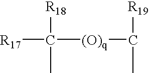

in which if q=0, R₁₇, R₁₈ and R₁₉, which are identical or different, each represent a hydrogen atom, a methyl, hydroxyl, acetoxy or amino residue, a monoalkylamine residue or a dialkylamine residue optionally interrupted by one or more nitrogen atoms and/or optionally substituted with one or more amine, hydroxyl, carboxyl, alkylthio or sulphonic groups, or an alkylthio residue whose alkyl group carries an amino residue, at least one of the R₁₇, R₁₈ and R₁₉ radicals being in this case a hydrogen atom;

or when q=1, R₁₇, R₁₈ and R₁₉ each represent a hydrogen atom, as well as the salts formed by these compounds with bases or acids.

(6) The polymers corresponding to the general formula (V) are described, for example, in French Patent 1,400,366:

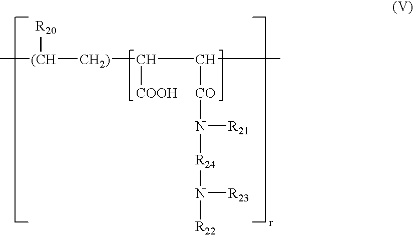

in which R₂₀ represents a hydrogen atom, a CH₃O, CH₃CH₂O or phenyl radical, R₂₁ denotes hydrogen or a lower alkyl radical such as methyl or ethyl, R₂₂ denotes hydrogen or a C₁-C₆ lower alkyl radical such as methyl or ethyl, R₂₃ denotes a C₁-C₆ lower alkyl radical such as methyl or ethyl or a radical corresponding to the formula: —R₂₄—N(R₂₂)₂, R₂₄ representing a group —CH₂—CH₂—, —CH₂—CH₂—CH₂— or —CH₂—CH(CH₃)—, R₂₂ having the above-mentioned meanings.

(7) The polymers derived from the N-carboxyalkylation of chitosan such as N-carboxymethyl chitosan or N-carboxybutyl chitosan sold under the name “EVALSAN” by the company JAN DEKKER.

(8) Amphoteric polymers of the -D-X-D-X- type chosen from:

a) the polymers obtained by the action of chloroacetic acid or sodium chloroacetate on the compounds containing at least one unit of formula:

-D-X-D-X-D- (VI)

where D denotes a radical

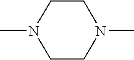

and X denotes the symbol E or E′, E or E′, which are identical or different, denote a bivalent radical which is an alkylene radical with a linear or branched chain containing up to 7 carbon atoms in the principal chain which is unsubstituted or substituted with hydroxyl groups and which may contain, in addition, oxygen, nitrogen or sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, or hydroxyl, benzylamine, amine oxide, quaternary ammonium, amide, imide, alcohol, ester and/or urethane groups.

b) The polymers of formula:

-D-X-D-X- (VI′)

where D denotes a radical

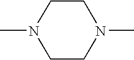

and X denotes the symbol E or E′ and, at least once, E′; E having the above-indicated meaning and E′ is a bivalent radical which is an alkylene radical with a linear or branched chain having up to 7 carbon atoms in the principal chain, which is unsubstituted or substituted with one or more hydroxyl radicals and containing one or more nitrogen atoms, the nitrogen atom being substituted with an alkyl chain optionally interrupted by an oxygen atom and necessarily containing one or more carboxyl functional groups or one or more hydroxyl functional groups and betainized by reaction with chloroacetic acid or sodium chloroacetate.

(9) The (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers may also contain other vinyl comonomers such as vinylcaprolactam.

Among the above-described amphoteric fixing polymers, those which are most particularly preferred according to the invention are those of the family (3) such as the copolymers whose CTFA name is Octylacrylamide/acrylates/butylaminoethyl methacrylate copolymer such as the products sold under the names AMPHOMER®, AMHOMER® LV 71 or LOVOCRYL® 47 by the company NATIONAL STARCH and those of the family (4) such as the methyl methacrylate/methyl dimethylcarboxymethylammonioethyl methacrylate copolymers, for example, sold under the name DIAFORMER Z301 by the company SANDOZ.

The nonionic fixing polymers which can be used according to the present invention are chosen, for example, from

polyalkyloxazolines

vinyl acetate homopolymers

vinyl acetate copolymers such as, for example, copolymers of vinyl acetate and acrylic ester, copolymers of vinyl acetate and ethylene, or copolymers of vinyl acetate and maleic ester, for example dibutyl maleate;

acrylic ester homopolymers and copolymers such as, for example, copolymers of alkyl acrylates and alkyl methacrylates such as the products provided by the company ROHM & HAAS under the names PRIMAL® AC-261 K and EUDRAGIT® NE 30 D, by the company BASF under the name 8845, by the company HOECHST under the name APPRETAN® N9212;

copolymers of acrylonitrile and of a nonionic monomer chosen, for example, from butadiene and alkyl (meth)acrylates; there may be mentioned the products provided under the name CJ 0601 B by the company ROHM & HAAS;

styrene homopolymers

styrene copolymers such as, for example, copolymers of styrene and alkyl (meth)acrylate such as the products MOWILITH® LDM 6911, MOWILITH® DM 611 and MOWILITH® LDM 6070 provided by the company HOECHST, the products RHODOPAS® SD 215 and RHODOPAS® DS 910 provided by the company RHONE POULENC; copolymers of styrene, alkyl methacrylate and alkyl acrylate; copolymers of styrene and butadiene; or copolymers of styrene, butadiene and vinylpyridine

polyamides;

vinyllactam homopolymers different from the vinylpyrrolidone homopolymers, such as polyvinylcaprolactam marketed under the name Luviskol® PLUS by the company BASF and

vinyllactam copolymers such as the poly(vinylpyrrolidone/vinyllactam) copolymers sold under the trade name Luvitec® VPC 55K65W by the company BASF, the poly(vinylpyrrolidone/vinyl acetate) copolymers such as those marketed under the name PVPVA® S630L by the company ISP, Luviskol® VA 73, VA 64, VA 55, VA 37 and VA 28 by the company BASF; and the poly(vinylpyrrolidone/vinyl acetate/vinyl propionate) terpolymers such as, for example, that marketed under the name Luviskol® VAP 343 by the company BASF.

The alkyl groups of the above-mentioned nonionic polymers preferably have from 1 to 6 carbon atoms.

According to the invention, it is also possible to use fixing polymers of the graft silicone type, comprising a polysiloxane portion and a portion consisting of a non silicone organic chain, one of the two portions constituting the principal chain of the polymer and the other being grafted on the said principal chain.

These polymers are for example described in Patent Applications EP-A-0 412 704, EP-A-0 412 707, EP-A-0 640 105 and WO 95/00578, EP-A-0 582 152 and WO 93/23009 and patents U.S. Pat. No. 4,693,935, U.S. Pat. No. 4,728,571 and U.S. Pat. No. 4,972,037.

These polymers may be amphoteric, anionic or nonionic, and they are preferably anionic or nonionic.

Such polymers are, for example, the copolymers which can be obtained by radical polymerization from the mixture of monomers consisting of:

a) 50 to 90% by weight of tert-butyl acrylate,

b) 0 to 40% by weight of acrylic acid,

c) 5 to 40% by weight of a silicone macromer of formula

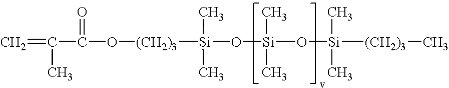

where v is a number ranging from 5 to 700, the percentages by weight being calculated relative to the total weight of the monomers.

Other examples of graft silicone polymers are in particular polydimethylsiloxanes (PDMS) onto which there are grafted, via a connecting group of the thiopropylene type, mixed polymer units of the poly((meth)acrylic acid) type and of the poly(alkyl (meth)acrylate) type, and polydimethylsiloxanes (PDMS) onto which there are grafted, via a connecting group of the thiopropylene type, polymer units of the poly(isobutyl (meth)acrylate) type.

As other type of silicone fixing polymers, there may be mentioned the product Luviflex® Silk marketed by the company BASF.

It is also possible to use, as fixing polymers, functionalized or nonfunctionalized, silicone or non silicone, cationic, nonionic, anionic or amphoteric polyurethanes, or mixtures thereof.

The polyurethanes particularly targeted by the present invention are those described in Applications EP 0 751 162, EP 0 637 600, EP 0 648 485 and FR 2,743,297 of which the applicant is proprietor, and in Applications EP 0 656 021 and WO 94/03510 by the company BASF, and EP 0 619 111 by the company National Starch.

As polyurethanes which are particularly suitable in the present invention, there may be mentioned the products marketed under the names LUVISET PUR® and LUVISET® Si PUR by the company BASF.

The water-soluble or water-dispersible fixing polymer is in particular present in a quantity of between 0.05 and 30% by weight, preferably between 0.1 and 30% by weight, and still more preferably between 0.5 and 15% by weight relative to the total weight of the hair-styling composiiton.

The hair-styling composition contained in the device according to the invention has a water content preferably of between 30 and 99.9% by weight, better still between 50 and 99% by weight, and still more particularly between 75 and 98% by weight relative to the total weight of the said composition.

The hair-styling composition may additionally comprise additives such as silicones in soluble, dispersed or microdispersed form, treatment active agents, moisturizers such as glycerol, UV-screening agents, acids, bases, plasticizers, solubilizing agents, preservatives, vitamins, provitamins, colorants, pigments, anionic, cationic, nonionic or amphoteric surfactants, perfumes, corrosion inhibitors, and mixtures thereof.

Persons skilled in the art will be careful to choose the optional additives and their quantity so that they do not adversely affect the properties of the compositions of the present invention.

These additives are present in particular in the composition according to the invention in a quantity ranging from 0 to 20% by weight relative to the total weight of the composition.

Preferably, the two-compartment aerosol device consists of an external aerosol can comprising an internal pouch which is hermetically sealed to a valve. The composition is introduced to the internal pouch and a compressed gas is introduced between the pouch and the can at a sufficient pressure to cause the product to come out in the form of a spray through the orifice of a nozzle. Such a device is marketed under the name EP SPRAY by the company EP-SPRAY SYSTEM SA.

The aerosol devices of the invention are preferably hair lacquers.

The present invention also relates to a hair-styling method which consists in spraying the hair-styling composition contained in the aerosol device according to the invention on hair which is wet or not.

The following examples are given by way of illustration of the present invention.

### EXAMPLE 1

A hair-styling composition was prepared from the following ingredients:

| | % by weight |
|---|---|
| Dimethylolpropionic acid/diisocyanate/neopentyl- | 5 AS * |
| glycol/polyesterdiols at 37% in water | |
| Demineralized water | qs 100 |

*Active substance

The composition as above-prepared is introduced into an aerosol dispenser marketed under the name EP SPRAY by the company EP SPRAY SYSTEM S.A. described above. A valve with the reference 6001 format D6 is fitted to a conventional aerosol can, and the diffuser is a swirl nozzle diffuser.

The pouch is filled with the composition as above-indicated. Compressed air is introduced between the pouch and the can.

The composition is sprayed onto dry hair. The spraying is carried out in the form of a gentle spray.

Good retention-exhibiting hair is obtained after drying.

### EXAMPLE 2

A second hair-styling composition was prepared from the following ingredients:

| | % by weight |
|---|---|
| Polyvinylcaprolactam at 27% in water | 2 AS |
| Tripropylene glycol monoethyl ether | 0.25 AS |
| Demineralized water | qs 100 |

*Active substance

The composition as above-prepared is introduced into an aerosol dispenser marketed under the name EP SPRAY as described in Example 1.

The composition is sprayed onto dry hair.

Results are obtained which are similar to those obtained in the case of Example 1.

### EXAMPLE 3

A hair-styling composition was prepared from the following ingredients:

| | % by weight |
|---|---|
| Vinylpyrrolidone/vinyllactam copolymer at 30% in | AS |
| water | |
| Demineralized water | qs 100 |

*Active substance

The composition as above-prepared is introduced into an aerosol dispenser marketed under the name EP SPRAY as described in Example 1.

The composition is sprayed onto dry hair.

Results are obtained which are similar to those obtained in the case of Example 1.

1. Two-compartment aerosol device, comprising:
a) in a first compartment, a hair-styling aqueous composition comprising less than 5% by weight, relative to the total weight of the said composition, of C₁-C₄ monohydric alcohols, and at least one water-soluble or water-dispersible fixing polymer, and
b) in a second compartment, a compressed gas chosen from air, nitrogen, carbon dioxide and mixtures thereof,
with the proviso that the water-soluble or water-dispersible fixing polymer is not a vinylpyrrolidone homopolymer.

2. Aerosol device according to claim 1, characterized in that the compressed gas is air.

3. Aerosol device according to claim 1 or 2, characterized in that the pressure of the compressed gas is between 1 and 12 bar.

4. Aersol device according to claim 3, characterized in that the pressure of the compressed gas is between 9 and 11 bar.

5. Aerosol device according to any one of the preceding claims, characterized in that the water-soluble or water-dispersible fixing polymer is chosen from cationic, anionic, amphoteric, and nonionic fixing polymers, and mixtures thereof.

6. Aerosol device according to claim 5, characterized in that the water-soluble or water-dispersible fixing polymer is cationic and is chosen from homopolymers or copolymers of acrylic or methacrylic esters or amides with amino functional groups, cationic polysaccharides, quaternary copolymers of vinylpyrrolidone and vinylimidazole, and chitosans.

7. Aerosol device according to claim 5, characterized in that the water-soluble or water-dispersible fixing polymer is anionic and is chosen from homopolymers or copolymers of acrylic and methacrylic acids or their salts, copolymers of crotonic acid, copolymers of monounsaturated C₄-C₈ carboxylic acids or anhydrides, polyacrylamides with carboxylate groups, homopolymers or copolymers with sulphonic groups, anionic polyurethanes, and anionic graft silicone polymers.

8. Aerosol device according to claim 5, characterized in that the water-soluble or water-dispersible fixing polymer is amphoteric and is chosen from copolymers with acidic vinyl units and basic vinyl units, crosslinked and acylated polyaminoamides, polymers with zwitterionic units, polymers derived from chitosan, modified (C₁-C₅)alkyl vinyl ether/maleic anhydride copolymers, amphoteric polyurethanes and amphoteric graft silicone polymers.

9. Aerosol device according to claim 5, characterized in that the water-soluble or water-dispersible fixing polymer is nonionic and is chosen from polyalkyloxazolines, homopolymers and copolymers of vinyl acetate, homopolymers and copolymers of acrylic esters, acrylonitrile copolymers, styrene homopolymers and copolymers, polyamides, vinyllactam homopolymers which are different from the vinylpyrrolidone homopolymers, vinyllactam copolymers, nonionic polyurethanes, and nonionic graft silicone polymers.

10. Aerosol device according to any one of the preceding claims, characterized in that the water-soluble or water-dispersible fixing polymer is present in a quantity of between 0.05 and 30% by weight relative to the total weight of the hair-styling composition.

11. Aerosol device according to claim 10, characterized in that the water-soluble or water-dispersible fixing polymer is present in a quantity of between 0.1 and 30% by weight relative to the total weight of the hair-styling composition.

12. Aerosol device according to any one of the preceding claims, characterized in that the hair-styling composition has a water content of between 30 and 99.9% by weight, relative to the total weight of the said hair-styling composition.

13. Aerosol device according to claim 12, characterized in that the water content is between 50 and 99% by weight relative to the total weight of the said composition.

14. Aerosol device according to any one of the preceding claims, characterized in that the hair-styling composition additionally comprises additives chosen from silicones in soluble, dispersed or microdispersed form, treatment active agents, moisturizers, UV-screening agents, acids, bases, plasticizers, solubilizing agents, preservatives, vitamins, provitamins, colorants, pigments, anionic, cationic, nonionic or amphoteric surfactants, perfumes, corrosion inhibitors, and mixtures thereof.

15. Aerosol device according to any one of the preceding claims, characterized in that it constitutes a hair lacquer.

16. Hair-styling method, characterized in that the hair-styling composition contained in the aerosol device according to the invention is sprayed onto hair which is wet or not.
